(19) United States
(12) Patent Application Publication
(10) Pub. No.: US 2008/0221573 A1
(43) Pub. Date: Sep. 11, 2008

(54) Adjustment Devices For External Fixators And Related Methods

(75) Inventor: David Kumhyr, Austin, TX (US)

Correspondence Address: LATHROP & GAGE LC, 4845 PEARL EAST CIRCLE, SUITE 300, BOULDER, CO, 80301, US

(73) Assignee: THINKVILLAGE, LLC, Niwot, CO (US)

(21) Appl. No.: 12/042,846

(22) Filed: Mar. 5, 2008

Related U.S. Application Data
(60) Provisional application No. 60/893,077, filed on Mar. 5, 2007.

(51) Int. Cl. A61B 17/00

(52) U.S. Cl. 606/59; 606/54; 606/57

(57) ABSTRACT

An adjustment device for an external fixator, which has an elongated fixator body connected to bone-pin clamps at proximal and distal ends of the elongated fixator body is disclosed. The adjustment device includes at least one adaptor for mounting to at least one of the bone-pin clamps. The adaptor stabilizes the bone-pins during an adjustment of the fractured bone to lessen the amount of pain experienced by a patient. An adjustment knob may be coupled to the adaptor and used to tighten or loosen a portion of the bone-pin clamp that facilitates rotation of the bone-pin clamp.

### RELATED APPLICATIONS

This application claims benefit of provisional U.S. Patent Application Ser. No. 60/893,077, filed Mar. 5, 2007, the disclosure of which is incorporated herein by reference.

### BACKGROUND

When bones break or fracture, extra support may be needed to hold them in the proper position until they are healed. An external fixator—a system of metal pins, rods, and clamps—can provide this extra support.

Metal pins of the external fixator are inserted into the fractured bone during an operation. The pins are placed on either side of the fracture site and an elongated fixator body, which is disposed outside of the patient's body, connects to the pins and provides stability. Typically, the external fixator remains in the fractured bone for six weeks while healing progresses. During this period, joint motion tends to become restricted due to binding of soft tissue to the pins of the external fixator. As a result, the patient may lose range of motion and extensive physical therapy may be required to regain full use of the injured limb.

In an attempt to shorten the amount of time a patient spends in physical therapy, an orthopedic surgeon may adjust the distraction, compression, translation, flexion, or extension of the external fixator throughout the healing period. This adjustment is generally accomplished using standard carpentry tools, such as screw drivers and box wrenches. These tools are effective for making coarse adjustments, but they provide no indication of the amount or rate of change. Adjustments are therefore arbitrary and painful, due to the amount of torque applied directly upon the pins which are embedded in the patient's bone.

FIG. 1 shows an external fixator 100 which enables an orthopedic surgeon to set and secure a fractured bone 108. Bone-pins 101 of external fixator 100 are driven into fractured bone 108 on either side of a fracture site 104 using surgical techniques known in the art, e.g., drilling. A pair of bone-pins 101 is secured to a clamp 102 by a screw 103 to form an assembly referred to herein as a bone-pin clamp 107. Multiple bone-pin clamps 107 are secured to elongated fixator body 106 by connectors 105 that provide for translation along elongated fixator body 106 in the direction of arrow A. Bone-pin clamp 107 may also rotate about a pivot 109, in the direction of arrow B, when screw 103 is loosened. Thus, the action of loosening screw 103, which is in direct contact with bone-pin clamp 107, using carpentry tools with long lever arms inflicts great pain on the patient due to the amount of torque applied directly to the bone-pins embedded in the patient's bone.

### SUMMARY

In one embodiment, an adjustment device for an external fixator is disclosed. The external fixator has an elongated fixator body connected to bone-pin clamps at proximal and distal ends of the elongated fixator body. The adjustment device includes at least one adaptor for mounting to at least one of the bone-pin clamps and at least one adjustment knob coupled to the adaptor and engaging a portion of the bone-pin clamp.

In one embodiment, an improvement to an external fixator having an elongated fixator body connected to bone-pin clamps at proximal and distal ends of the elongated fixator body includes an adjustment device. The adjustment device has at least one adaptor for mounting to at least one of the bone-pin clamps and at least one adjustment knob coupled to the adaptor for engaging a portion of the bone-pin clamp.

In one embodiment, a method is disclosed for operating an adjustment device for an external fixator. The external fixator has an elongated fixator body connected to bone-pin clamps at proximal and distal ends of the elongated fixator body. The method includes mounting at least one adaptor to at least one of the bone-pin clamps and coupling at least one adjustment knob to the adaptor.

In one embodiment, an adjustment device for an external fixator is disclosed. The external fixator has an elongated fixator body connected to bone-pin clamps at proximal and distal ends of the elongated fixator body. The adjustment device includes at least one adaptor for mounting to at least one of the bone-pin clamps, where the adaptor comprises a conduit that provides access to the bone-pin clamp.

### BRIEF DESCRIPTION OF THE FIGURES

FIG. 1 shows an exemplary external fixator.

FIG. 2 shows an exemplary adjustment device for an external fixator, according to an embodiment.

FIG. 3A shows an exemplary top or bottom plan view of an adaptor of the adjustment device of FIG. 2.

FIG. 3B shows an exemplary back plan view of the adaptor of FIG. 3A.

FIG. 4 shows an exemplary front plan view of an adaptor having degree markings.

FIG. 5 shows an exemplary side plan view of an adjustment knob.

FIGS. 6A and 6B illustrate rotation of a bone-pin clamp of an external fixator.

FIG. 7 shows an adaptor for use without an adjustment knob, according to an embodiment.

FIG. 8 shows an adaptor having an inclinometer, according to an embodiment.

FIG. 9 shows a method of operating an adjustment device, according to an embodiment.

### DETAILED DESCRIPTION OF THE FIGURES

Reference will now be made to the attached drawings, where multiple elements within a figure may not be labeled for the sake of clarity, and the figures may not be drawn to scale.

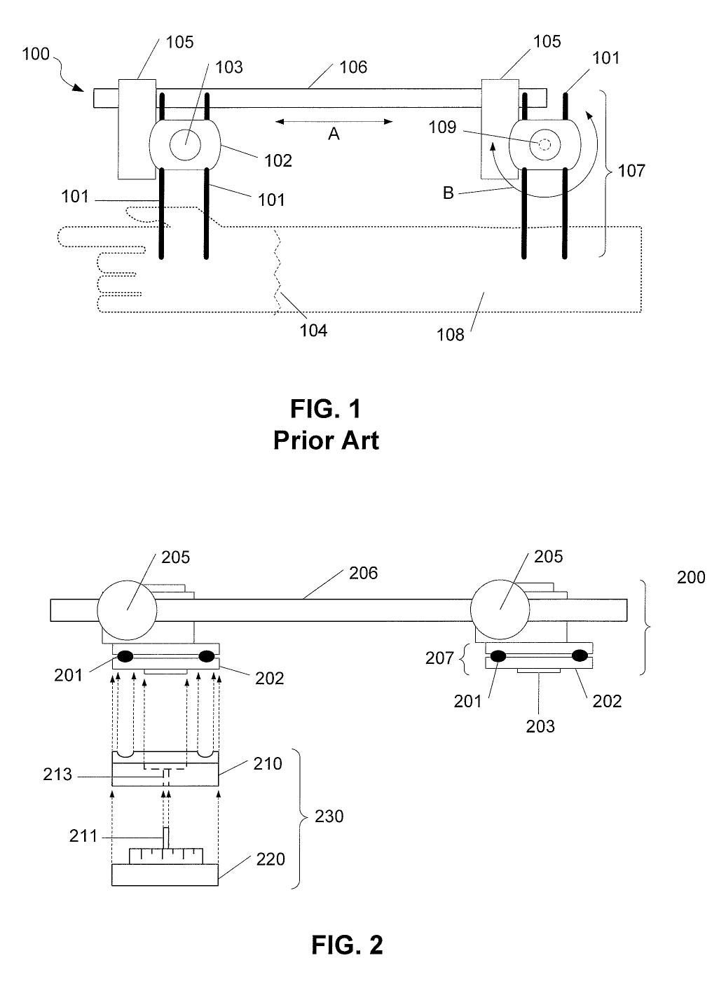

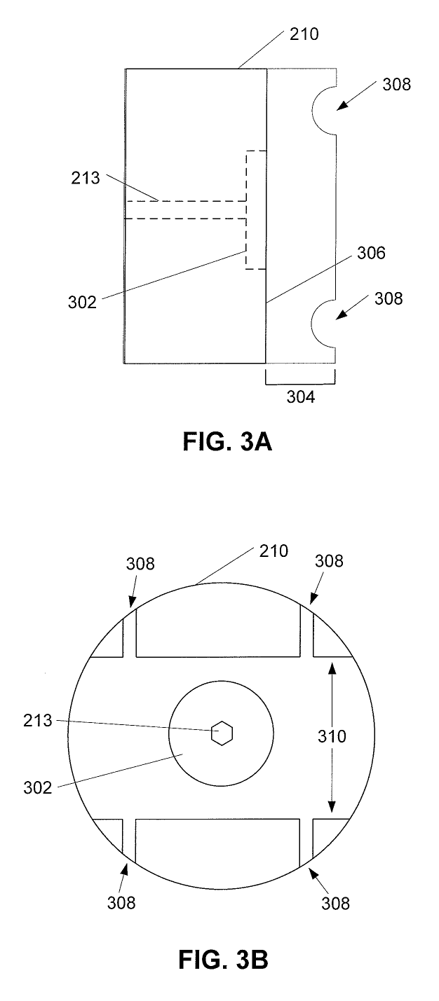

FIG. 2 shows an external fixator 200, which may represent external fixator 100 of FIG. 1, and an exemplary adjustment device 230. Adjustment device 230 includes an adaptor 210, described in further detail in FIGS. 3A and 3B, and an adjustment knob 220. External fixator 200 has an elongated fixator body 206, such as elongated fixator body 106 of FIG. 1; connectors 205, such as connectors 105 of FIG. 1; and at least one bone-pin clamp 207, such as bone-pin clamp 107 of FIG. 1. Bone-pin clamp 107 includes a clamp 202, such as clamp 102 of FIG. 1; a pair of bone-pins 201, such as bone-pins 101 of FIG. 1; and a screw 203, such as screw 103 of FIG. 1. Adaptor 210 may be mated with bone-pin clamp 207 in a lock-and-key fashion, where a back side of adapter 210 contains recesses for receiving a head of screw 203, clamp 202, and sections of bone-pins 201 that protrude from clamp 202. Adjustment knob 220 couples to adaptor 210 by insertion of key 211 into a key slot 213. Key slot 213 provides access for key 211 to engage screw 203. Thus, a surgeon, or other person, adjusting external fixator 200 with adjustment device 230, will hold adaptor 210 in a fixed position while turning adjustment knob 220. The fixed position of adaptor 210 insures that bone-pins 201 are stabilized while screw 203 is loosened, thereby lessening the amount of pain experienced by a patient.

The above-described external fixator and adjustment device components may be fabricated, for example, from one or more materials selected from stainless steel, ceramics, metals, metal alloys, plastics, titanium, and combinations thereof. For example, adjustment knob 220 may be titanium with a plastic or rubber coating forming a non-slip grip. In another example, texturing of metal, plastic, rubber, or ceramic surfaces may provide increased friction for effective gripping.

FIG. 3A shows a top or bottom view of adaptor 210 of adjustment device 230 configured for mounting to an external fixator (e.g., external fixator 100 of FIG. 1 or external fixator 200 of FIG. 2). FIG. 3A may be viewed in combination with FIG. 3B, which shows a back plan view of adaptor 210. Adaptor 210 has a key slot 213 for receiving key 211 of adjustment knob 220. Key slot 213 may vary in shape and size depending on the type of key 211 necessary to engage screw 203. At a distal end of key slot 213, a first recess 302 receives the head of screw 203. A second recess (310, FIG. 3B) within area 304 receives clamp 202, which abuts wall 306 when adaptor 210 is snuggly engaged with bone-pin clamp 207. Adaptor 210 also has a set of recesses 308, for receiving portions of bone-pins 201 that protrude from clamp 202.

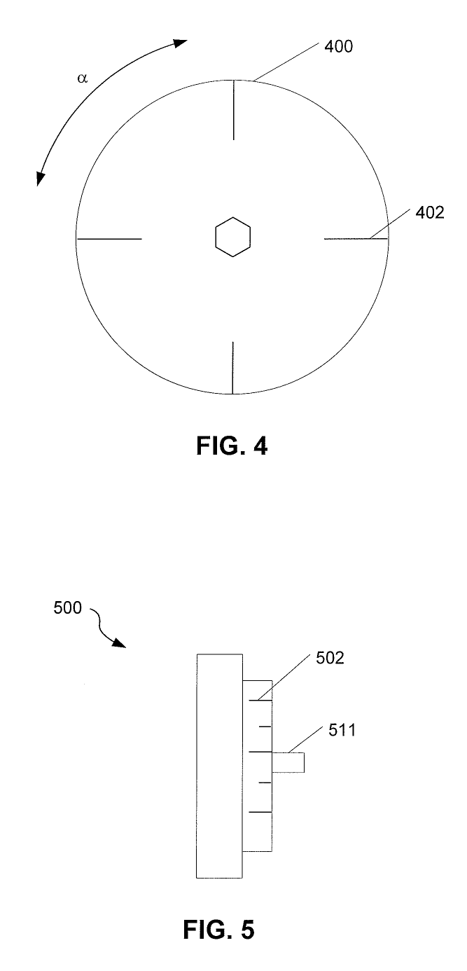

FIG. 4 shows a front plan view of an adaptor 400 having graduated markings 402. Graduated markings 402 may, for example, be degree markings or one or more reference marks to indicate a degree of rotation, α, relative to another set of graduated markings (e.g., 502, FIG. 5). The presence of one or more sets of graduated markings 402, 502 allows for measurement of a degree of rotation and/or rate of rotation, of a bone-pin clamp (e.g., bone-pin clamp 107 of FIG. 1 or bone-pin clamp 207 of FIG. 2) during adjustment of an external fixator (e.g., external fixator 100 of FIG. 1 or external fixator 200 of FIG. 2). It will be appreciated that more or fewer markings than illustrated may be used, and numbers corresponding to a degree of rotation may or may not be present.

FIG. 5 shows a side plan view of an adjustment knob 500. Adjustment knob 500 has graduated markings 502, which are shown as disposed on a side of adjustment knob 500. It will be appreciated that graduated markings 502 may also, or alternatively, be disposed on a front face of adjustment knob 500. Graduated markings 502 may be degree markings or one or more reference marks to indicate a degree of rotation relative to another set of graduated markings (e.g., 402, FIG. 4).

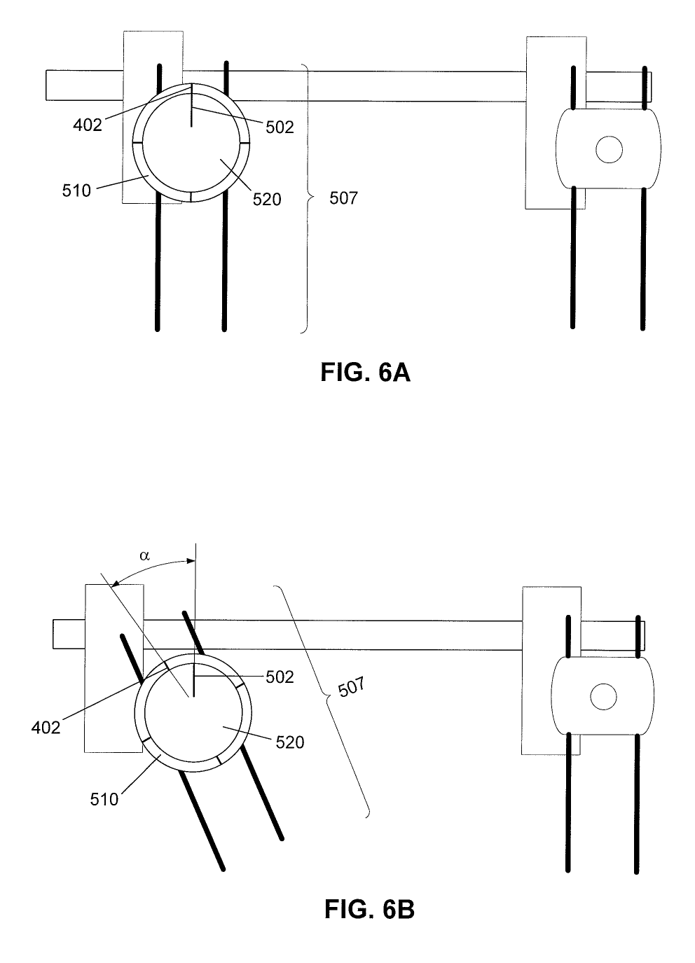

FIGS. 6A and 6B, when viewed together, illustrate rotation of a bone-pin clamp 507 about a pivot (e.g., pivot 109, FIG. 1). FIG. 6A shows bone-pin clamp 507 in a first, unrotated position. Graduated markings 402 and 502 are aligned, indicating that the degree of rotation, α, is zero degrees. FIG. 6B shows an exemplary result of an adjustment performed using the disclosed adjustment device (e.g., adjustment device 230 of FIG. 2). Adaptor 510 remains in its initial position relative to bone-pin clamp 507, and rotates along with bone-pin clamp 507. Adjustment knob 520 is stationary during rotation of bone-pin clamp 507, and marks the starting position. The angular displacement between mark 502 of adjustment knob 520 and mark 402 of adaptor 510 allows a surgeon to identify the degree of rotation, α, of bone-pin clamp 507. When a desired degree of rotation is reached, the screw of bone-pin clamp 507 is tightened using adjustment knob 520.

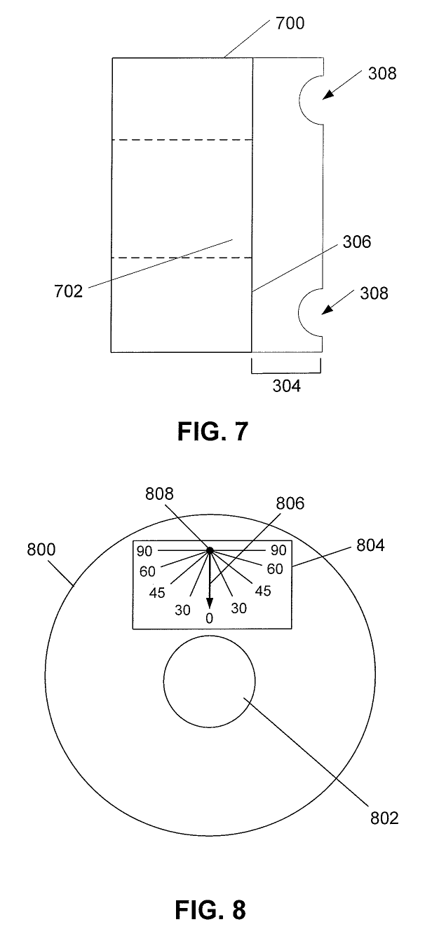

FIG. 7 shows an adaptor 700, which may be used to stabilize a bone-pin clamp (e.g., bone-pin clamp 107 of FIG. 1 or bone-pin clamp 207 of FIG. 2) while allowing access to the bone-pin clamp screw (e.g., screw 103 of FIG. 1 or screw 203 of FIG. 2). According to this embodiment, it is not necessary to use an adjustment knob to loosen the bone-pin clamp screw. Adaptor 700 has recesses 308 and 310 similar to those shown in FIGS. 3A and 3B. However, recess 302 of FIGS. 3A and 3B has been enlarged to form a conduit 702. Conduit 702 may, for example, receive a screw driver head that engages the bone-pin clamp screw.

FIG. 8 shows an adaptor 800 having an inclinometer 804 fixed thereon. Inclinometer 804 comprises a pointer 806 that rotates about a pivot 808. An angle of rotation, α, is indicated on a scale of inclinometer 804 when adaptor 800 rotates. Adaptor 800 includes a conduit 802, similar to conduit 702 of FIG. 7, that allows adaptor 800 to be used without an adjustment knob. Inclinometer 804 therefore does not impede mating of an adjustment knob to adaptor 800.

It will be appreciated that other devices for measuring an angle of rotation, tilt angle or incline may be used with the adjustment devices described herein. For example, electronic inclinometers may be used.

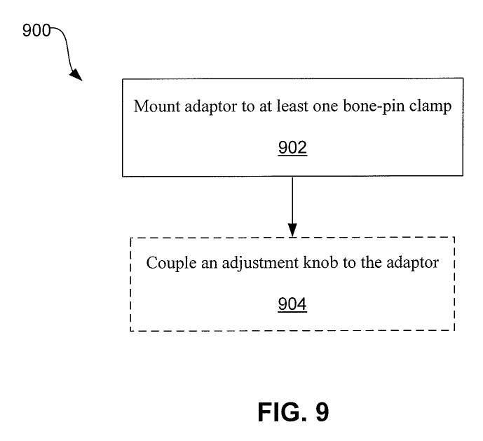

FIG. 9 shows a method 900 for operating an adjustment device (e.g., adjustment device 230 of FIG. 2). Method 900 is performed on an external fixator for selective periodic distraction of a fractured bone that has been set for retention, such as external fixator 100 of FIG. 1 or external fixator 200 of FIG. 2. In step 902, an adaptor (e.g., adaptor 210 of FIG. 2) is mounted to at least one bone-pin clamp (e.g., bone-pin clamp 207 of FIG. 2).

In one embodiment, an adjustment knob (e.g., adjustment knob 230 of FIG. 2) is coupled to the adaptor in step 904.

Changes may be made in the above methods and systems without departing from the scope hereof. It should thus be noted that the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall there between.

What is claimed is:

1. An adjustment device for an external fixator, the external fixator having an elongated fixator body connected to bone-pin clamps at proximal and distal ends of the elongated fixator body, the adjustment device comprising:
at least one adaptor for mounting to at least one of the bone-pin clamps; and
at least one adjustment knob for coupling to the adaptor and engaging a portion of the bone-pin clamp.

2. The device of claim 1, wherein the adaptor comprises graduated markings.

3. The device of claim 1, wherein the adaptor comprises a key slot for receiving a key of the adjustment knob.

4. The device of claim 1, wherein the adjustment knob comprises graduated markings.

5. The device of claim 1, wherein the adjustment knob comprises an inclinometer.

6. The device of claim 1, wherein the adjustment knob comprises a key for engaging the bone-pin clamp.

7. The device of claim 1, wherein the adjustment device is fabricated from a material selected from the group consisting of stainless steel, ceramics, metals, metal alloys, plastics, titanium, and combinations thereof.

8. An improvement to an external fixator having an elongated fixator body connected to bone-pin clamps at proximal and distal ends of the elongated fixator body, the improvement comprising:
an adjustment device having:
at least one adaptor for mounting to at least one of the bone-pin clamps; and
at least one adjustment knob coupled to the adaptor for engaging a portion of the bone-pin clamp.

9. The device of claim 8, wherein the adaptor comprises graduated markings.

10. The device of claim 8, wherein the adaptor comprises a key slot for receiving a key of the adjustment knob.

11. The device of claim 8, wherein the adjustment knob has graduated markings.

12. The device of claim 8, wherein the adjustment knob comprises a key for engaging the bone-pin clamp.

13. The device of claim 8, wherein the adjustment device is fabricated from a material selected from the group consisting of stainless steel, ceramics, metals, metal alloys, plastics, titanium, and combinations thereof.

14. A method for operating an adjustment device for an external fixator, the external fixator having an elongated fixator body connected to bone-pin clamps at proximal and distal ends of the elongated fixator body, the method comprising:
mounting at least one adaptor to at least one of the bone-pin clamps; and
coupling at least one adjustment knob to the adaptor.

15. The method of claim 14, further comprising rotating the bone-pin clamps during an adjustment of a fractured bone.

16. The method of claim 14, further comprising identifying a degree of rotation of the bone-pin clamp.

17. An adjustment device for an external fixator, the external fixator having an elongated fixator body connected to bone-pin clamps at proximal and distal ends of the elongated fixator body, the adjustment device comprising:
at least one adaptor for mounting to at least one of the bone-pin clamps,
wherein the adaptor comprises a conduit that provides access to the bone-pin clamp.

18. The device of claim 17, wherein the adaptor comprises graduated markings.

19. The device of claim 17, wherein the adaptor comprises an inclinometer.

20. The device of claim 17, wherein the adjustment device is fabricated from a material selected from the group consisting of stainless steel, ceramics, metals, metal alloys, plastics, titanium, and combinations thereof.
